(19) United States
(12) Patent Application Publication
(10) Pub. No.: US 2013/0006045 A1
(43) Pub. Date: Jan. 3, 2013

(54) INCONTINENCE TREATMENT DEVICE WITH PLICATION MECHANISM

(75) Inventors: Sarah J. Deitch, Minneapolis, MN (US); Thomas Kubalak, Wayzata, MN (US); Mark A. Moschel, New Hope, MN (US); Thomas Broome, Chaska, MN (US)

(73) Assignee: COLOPLAST A/S, Humlebaek (DK)

(21) Appl. No.: 13/172,831

(22) Filed: Jun. 30, 2011

(30) Foreign Application Priority Data
Jun. 29, 2011 (DK) PA 2011 70337

(51) Int. Cl. A61F 2/04

(52) U.S. Cl. 600/30

(57) ABSTRACT

An incontinence treatment device includes a support body and a plication mechanism attached to the support body. The device includes a first and second arms separated by a support zone. The plication mechanism is attached to the support zone and has a strand providing first and second end portions. The strand is removably coupled with a first location of the support zone and removably coupled with a separate second location of the support zone. The separate second location is spaced apart from the first location of the support zone by a central region. The support body is configured to contact tissue around a urethra and tension applied to both of the first and second end portions moves the first location of the support zone and the second location of the support zone to remove slack in the support body relative to the tissue around the urethra.

### BACKGROUND

Urinary incontinence is the undesired leakage of urine from the bladder and this malady affects both men and women, with some estimates as high as 30% of the population experiencing some level of urinary incontinence.

Devices for treating urinary incontinence include slings, supports, and other scaffold-like devices that are implanted in a patient's body to support, elevate, or compress the urethra. A urethral support is a urinary incontinence treatment device that is surgically implanted under the urethra to inhibit urine from undesirably exiting the urethra, particularly during a provocative event such as when coughing or sneezing.

Other urinary incontinence treatment devices include artificial sphincters that selectively coapt the urethra and injected bulking liquids that are applied alongside a layer of the urethra to support or partially coaptate the urethra.

Improved incontinence treatment methods and devices would be welcomed by both the patient and the surgical staff.

### SUMMARY

One aspect provides an incontinence treatment device including a support body and a plication mechanism. The support body includes a first arm and a second arm extending from the support body with the first arm separated from the second arm by a support zone. The plication mechanism is attached to the support zone and includes a strand providing first and second end portions. The strand is removably coupled with a first location of the support zone and removably coupled with a separate second location of the support zone, where the separate second location is spaced apart from the first location of the support zone by a central region. The support body is configured to contact tissue around a urethra and tension applied to both of the first and second end portions moves the first location of the support zone and the second location of the support zone to remove slack in the support body relative to the tissue around the urethra.

One aspect provides a method of treating urinary incontinence including thrilling an incision and accessing tissue around a urethra of a patient. The method additionally includes passing a first transobturator arm of an incontinence treatment device through the incision and through a first obturator foramen of the patient and a second transobturator arm of the incontinence treatment device through the incision and through a second obturator foramen of the patient. The method additionally includes passing a first pubic arm of the incontinence treatment device through the incision to a location anterior a pubic bone of the patient and a second pubic arm of the incontinence treatment device through the incision to a location anterior a pubic bone of the patient. The method additionally includes applying tension to the first and second transobturator arms and the first and second pubic arms of the incontinence treatment device and tensioning a urethral support body of the incontinence treatment device against the tissue around the urethra of the patient. The method further includes pulling strands of a plication mechanism that is attached to the urethral support body and taking up slack in the urethral support body relative to the tissue around the urethra of the patient.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a top view of one embodiment of an incontinence treatment device including a plication mechanism attached to a support body.

FIG. 2 is a schematic representation of one embodiment of the incontinence treatment device illustrated in FIG. 1 representing the arms in an implanted configuration.

FIG. 3 is a schematic view of the incontinence treatment device illustrated in FIG. 2 with the plication mechanism activated to gather together excess material of the support body.

FIG. 4 is a block diagram of one embodiment of a method of treating urinary incontinence.

FIG. 5 and FIG. 6 are perspective schematic views of embodiments of the incontinence treatment device implanted in a male patient.

FIG. 7 is a top view of one embodiment of an incontinence treatment device including a plication mechanism attached to a support body.

FIG. 8 is a top view of one embodiment of an incontinence treatment device including a plication mechanism attached to a support body.

FIG. 9 is a schematic representation of one embodiment of the incontinence treatment device illustrated in FIG. 8 with the arms in an implanted configuration.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

People suffering from urinary incontinence have a diminished ability to restrict the flow of urine through the urethra, usually due to a damaged or deficient urethral sphincter. The urethral sphincter is a collection of muscles that control the flow of urine from the bladder. These muscles envelop the urethra, and when contracted, seal the urethra shut. In general terms, each person has two urethral sphincters: an internal sphincter muscle of the urethra and an external sphincter muscle of the urethra. The internal sphincter muscle of the urethra is located near the junction of the urethra and the bladder. The external sphincter muscle of the urethra is located at the distal inferior end of the bladder in females and inferior to the prostate (at the level of the membranous urethra) in males.

The urethra is normally supported by connective and other tissues. The support provided by the connective tissues to the urethra can erode over time, giving rise to hyper-mobility of the urethra. Hyper-mobile urethras are susceptible to the undesirable leaking of urine during provocative events such as sneezing, laughing, or coughing which is sometimes referred to as stress urinary incontinence).

The implant described herein provides a urinary incontinence device that provides support to a patient's urethra. The device is configured to be tensioned over tissues surrounding the urethra, and the device allows excess material of a support body of the device to be gathered, folded, or plicated to provide a more comfortable implant having improved urethral support. For example, the tissue around the urethra provides a three-dimensional shape having curvature, typically, and both a longitudinal and a lateral direction. Placement of a support body of an incontinence treatment device against the complex curvature of the tissue around the urethra can give rise to a certain level of sagging of the support body away from the tissue. The implant described herein includes a plication mechanism that allows excess material of the support body to be gathered together to ensure an intimate fit of the support body against the urethral tissue.

Plication means to fold a portion or portions of a material together or to gather portions of a material together.

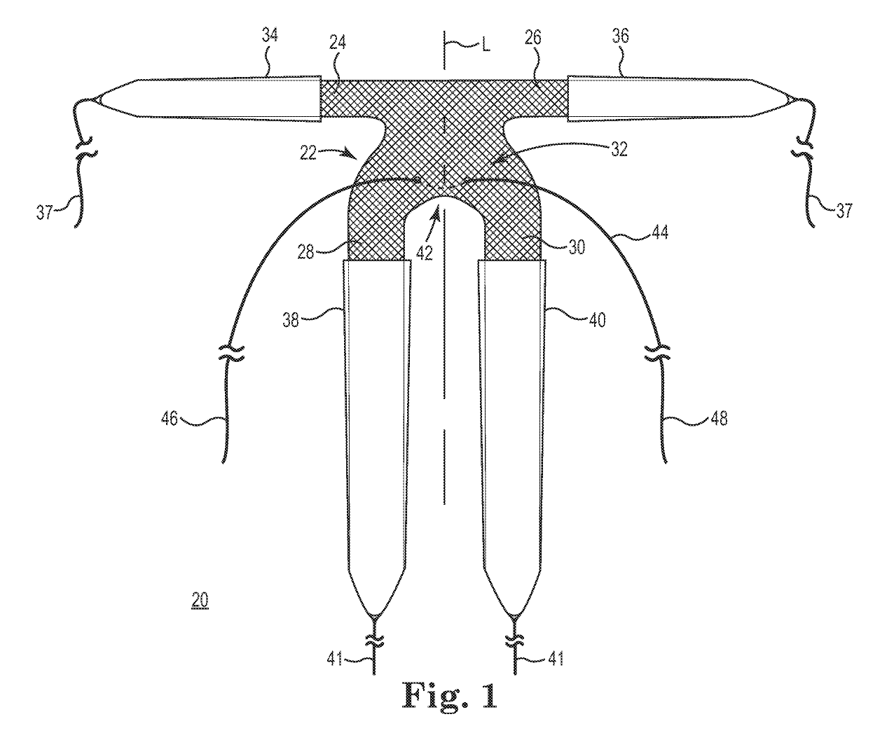

FIG. 1 is a top view of one embodiment of an incontinence treatment device 20 (device 20). The device 20 includes a support body 22 that defines a longitudinal axis L, arms 24, 26 extending from the support body 22 in a direction that is substantially orthogonal to the longitudinal axis L, arms 28, 30 extending from the support body 22 in a direction substantially parallel with the longitudinal axis L, and a plication mechanism 32 attached to the support body 22.

in one embodiment, the arms 24, 26 are provided as transobturator arms and each include a removable insertion sheath 34, 36, respectively, and a suture 37 attached to the end of each arm 24, 26 and each respective insertion sheath 34, 36. The transobturator arms 24, 26 are configured to pass through a membrane covering an obturator foramen, and the insertion sheaths 34, 36 are provided to allow the arms 24, 26 to pass through the membrane covering the obturator foramen in a way that reduces the curling of the edges of the arms 24, 26. The insertion sheaths 34, 36 are removable from the arms 24, 26 after the device 20 is implanted. The suture 37 is provided to guide the arms 24, 26 and the insertion sheath 34, 36, respectively, when implanting the device 20. For example, during implantation of the device 20 an introducer device is utilized to separately engage the suture 37 and pass the first and second arms 24, 26 through a respective one of the first and second obturator foramen of the patient.

In one embodiment, the arms 28, 30 are provided as pubic arms and each include a removable insertion sheath 38, 40, respectively, and a suture 41 attached to an end of each of the arms 28, 30 and each respective insertion sheath 38, 40. The pubic arms 28, 30 are configured to be passed subcutaneously within the patient, and the insertion sheaths 38, 40 are provided to allow the arms 28, 30 to pass under the tissue while reducing undesirable curling of the edges of the arms 28, 30. The suture 41 is provided to guide the subcutaneous placement of the arms 28, when implanting the device 20.

In one embodiment, the arms 24, 26 and 28, 30 are integrally formed with the support body 22 such that the support body 22 and the arms 24, 26 and 28, 30 are fabricated from a single unitary piece of material. In one embodiment, this single unitary piece material is a porous polymer mesh. In one embodiment, the arms 24, 26 and 28, 30 are separately attached to the support body 22 and are fabricated from a material that is different than the support body 22.

In one embodiment, the insertion sheaths 34, 36 and 38, 40 are provided as smooth polymer films and are fabricated in a tubular structure and so configured to be removably placed over each of the arms 24, 26 and 28, 30, respectively.

The sutures 37 and 41 are suitably selected from thermoplastic sutures, non-thermoplastic sutures, multi-filament sutures, or mono-filament sutures.

In one embodiment, the support body 22 is porous (having a plurality of pores or openings) and includes a support zone 42, with the plication mechanism 32 attached to the support zone 42. In one embodiment, the plication mechanism 32 is provided as a single strand 44 having end portions 46, 48 and is threaded through pores of the support zone 42. The support zone 42 is configured to be folded or collapsed when tension is provided to both of the first and second end portions 46, 48. The plication mechanism 32 is configured to be removable from the support body 22 when tension or a pulling force is provided to only one of the end portions 46, 48 of the strand 44.

In one embodiment, the support zone 42 is bisected by the longitudinal axis L and the plication mechanism 32 is attached to the support zone 42 in a symmetric manner such that the strand 44 is centered relative to the longitudinal axis L. In this manner, the plication mechanism 32 is a visual indicator that identifies a mid-line of the support zone 42 and serves as an aid in the implantation of the implant 20 in a centered and balanced location relative to the urethra. The plication mechanism 32 is thus symmetrically centered relative to the central axis L of the support zone 42 to provide a visual indicator for equal distribution of the support body 22 relative to a urethra of a patient.

The strand 44 of the plication mechanism 32 is suitably selected from suture material available from Teleflex, Limerick, Pa. or CP Medical, Portland, Oreg. Other suitable strands are available from Ethicon™, a J&J Company located in Somerville, N.J., and include resorbable and other sutures such as Monocryl™ (polyglycaprone 25) sutures, coated Vicryl™ (polyglactin 910) sutures, Ethicon Plus™ Sutures, or polydioxanone sutures as examples. Examples of suitable body-absorbable sutures are the Caprosyn™ Polysorb™, and Biosyn™ absorbable sutures available from Covidien, Mansfield, Mass.

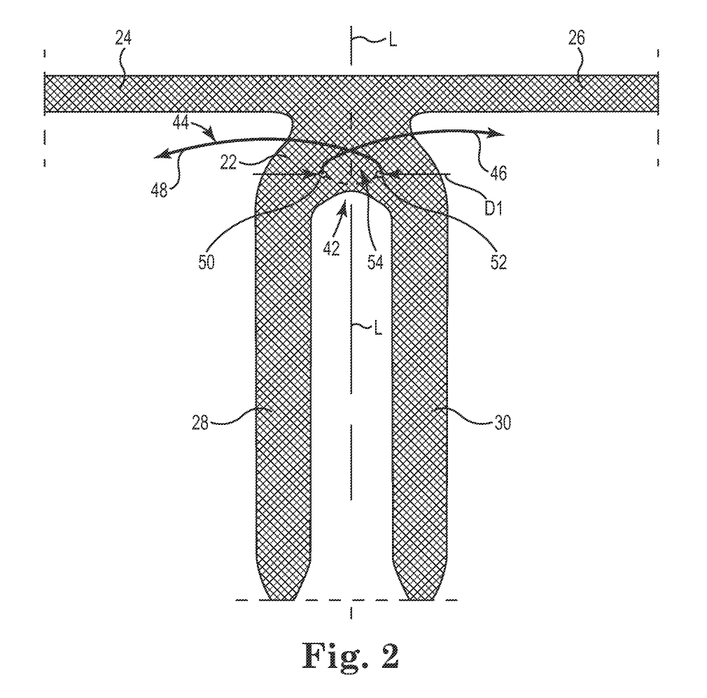

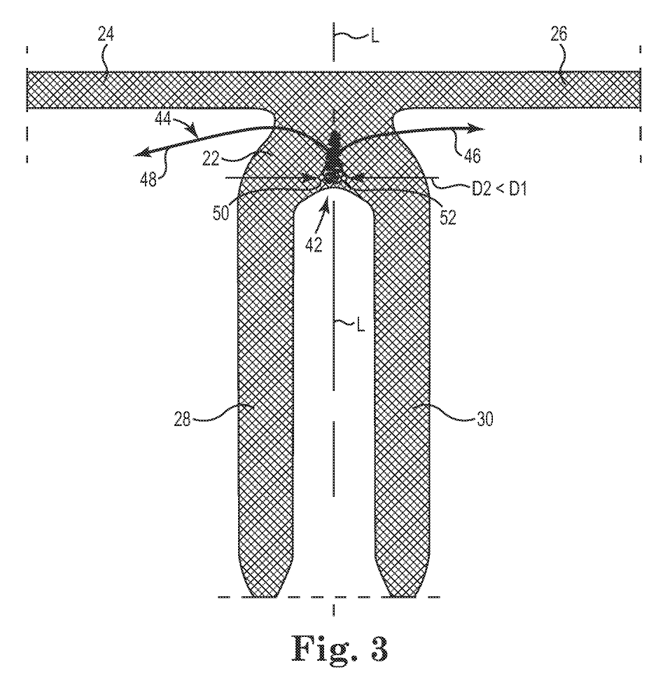

FIG. 2 and FIG. 3 are schematic representations of one embodiment of the incontinence treatment device illustrated in FIG. 1 with the arms shown in an implanted configuration. In particular, the schematic representations illustrate that the arms 24, 26 and 28, 30 are implanted in tissue and the sutures 37, 41 and the insertion sheaths 34, 36 and 38, 40 (FIG. 1) have been removed. It is to be understood that the arms 24, 26 and 28, 30 are under tension after being implanted in tissue.

In one embodiment, the support body 22 is substantially bisected by the longitudinal axis L, and the support zone 42 includes a first location 50 on a first side of the longitudinal axis L and a second location 52 on a second side of the longitudinal axis L generally opposite of the first side 50. The first location 50 and the second location 52 are generally spaced apart one from the other by a distance D1 within a central region 54 of the support zone 42.

With reference to FIG. 3, in one embodiment the end portions 46, 48 of the strand 44 are overlapped (right to left and left to right) such that when tension is applied to the end portions 46, 48 the first location 50 moves towards the longitudinal axis L and the second location 52 likewise moves toward the longitudinal axis L to reduce the distance from the distance D1 down to the distance D2 and thus compress or compact the central region 54 (FIG. 2). In this manner, activating the plication mechanism 32 operates to move the locations 50, 52 in a medial direction and take up any slack or excess material in the support zone 42 by folding or gathering or otherwise plicating the material and a support zone 42.

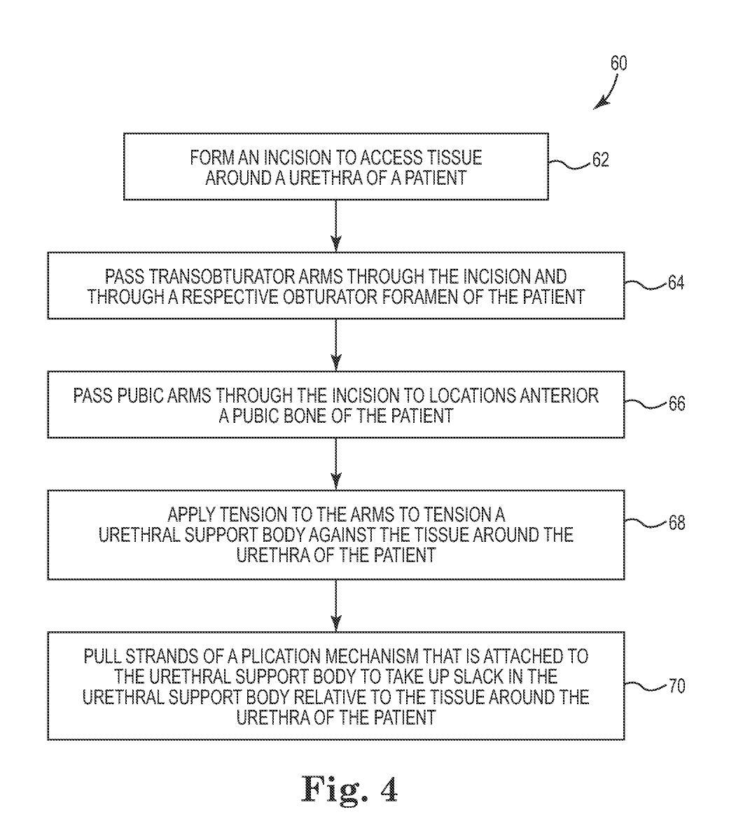

FIG. 4 is a block diagram of one embodiment of a method 60 of treating urinary incontinence. The method 60 is described with reference to FIG. 1 and includes at 62 forming an incision to access tissue around a urethra of the patient. The method includes at 64 passing the transobturator arms 24, 26 through the incision and through a respective obturator foramen of the patient. The method includes at 66 passing the pubic arms 28, 30 through the incision to locations that are anterior the pubic bone the patient (e.g., between the pubic bone and the skin surface of the patient). The method includes at 68 applying tension to the arms to tension the urethral support body 22 against the tissue around the urethra of the patient. The method additionally includes at 70 pulling the strands of the plication mechanism 32 that are attached to the urethral support body 22 to take up slack or excess material in the urethral support body relative to the tissue around the urethra of the patient.

Aspects of one suitable surgical procedure are described in the following paragraphs. The patient is typically placed in a dorsal lithotomy position with the legs positioned at about 90° and held in place by stirrups. The patient is catheterized, for example with a 14 French catheter. The surgeon makes a vertical perineal incision in the midline and dissects tissue to eventually isolate the ventral bulbous urethra and pubic rami while ensuring that the bulbospongiosus muscle around the urethra is intact. The surgeon will subsequently expose the bulbospongiosus muscle and take it off the perineal body to allow ventral urethral elevation compression by the support body 22.

The device 20 is placed by engaging the suture 37 attached to each one of the transobturator arms 24, 26 to a suitable introducer and passing each of the arms 24, 26 through the perineal incision from the medial to the lateral through the obturator foramen in what has become known as art “inside-out” maneuver.

A suitable introducer is employed that is passed from above the pubic symphysis and lateral to the midline of the pubic symphysis, downward subcutaneously until the introducer exits a perineal incision lateral to the urethra. The suture 41 is attached to the introducer. The introducer is withdrawn along its pathway subcutaneously from the perineal incision in an upward route that is anterior to the pubic bone until the introducer in the suture 41 exits the skin.

The arms 24, 26 are tensioned by pulling opposite ones of the suture 37. The arms 28, 30 are tensioned by pulling opposite ones of the suture 41. The surgeon may loosen the support body 22, if desired, by placing an instrument between the support body 22 and the tissue and employing the instrument to pull the support body 22 away from the tissue around the urethra to loosen the support body 22. When the surgeon determines that the device 20 is placed as desired, the sutures 37, 41 and the insertion sheaths 34, 36 and 38, 40 are removed. The arms 24, 26 and 28, 30 are trimmed level, or just below level, with the subcutaneous tissue.

The strand 44 of the plication mechanism 32 extends out of the perineal incision and affords the surgeon the option of plicating the support zone 42 to more snugly support the tissue around the urethra. For example, the surgeon will pull on the end portions 46, 48 of the plication mechanism 32 to gather the excess material in the support zone 42 tightly against the tissue around the urethra. The surgeon will terminate the strand 44, for example by tying the end portions 46, 48 and trimming as desired. In one embodiment, the surgeon may determine that the support zone 42 has been initially placed at a desirable supporting tension, in which case surgeon pulls on one of the end portions 46, 48 to remove the strand 44 from the support zone 42.

The device 20 is implanted as described above and the surgeon will typically close the perineal incision with multiple layers of closure and dose the incisions made in other portions of the skin with sutures.

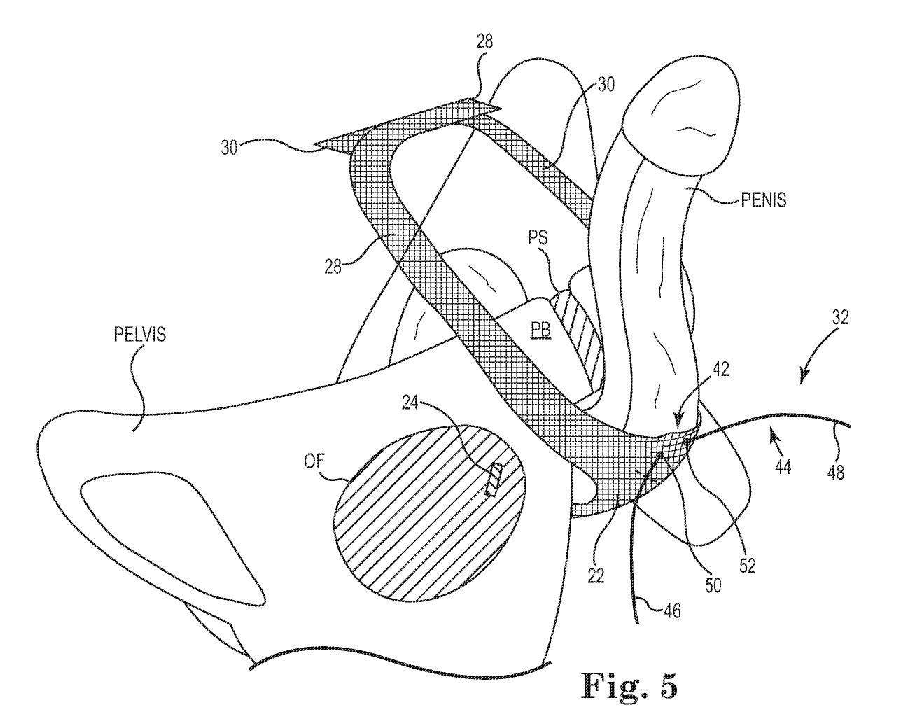

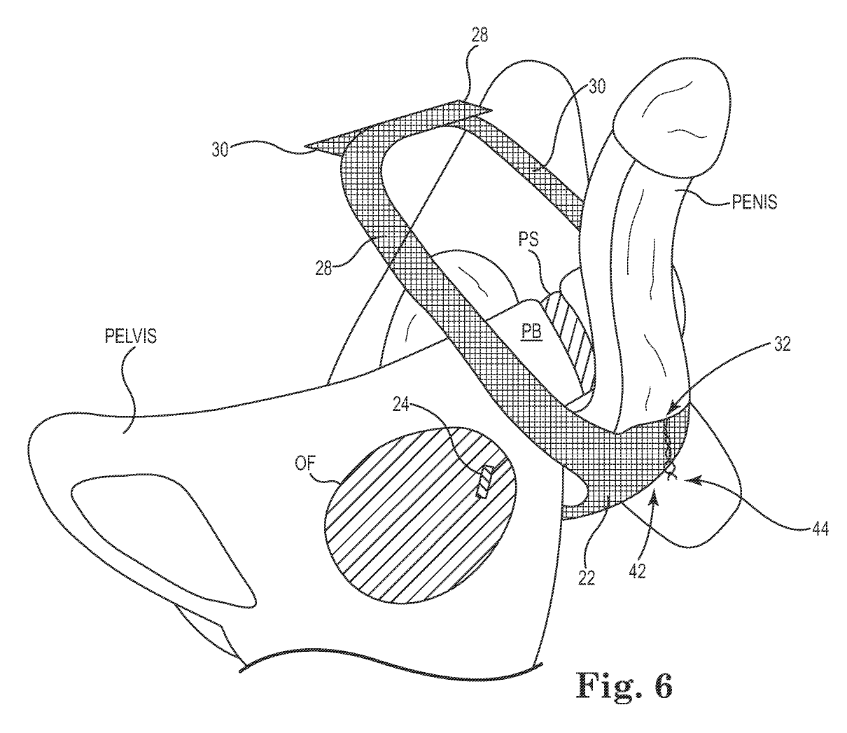

FIG. 5 and FIG. 6 are perspective schematic views of embodiments of the incontinence treatment device implanted in a male patient relative to a pelvis P as described above.

FIG. 5 illustrates the support zone 42 of the support body 22 after being implant against tissue that surrounds the urethra (not shown but within the penis). The transobturator arms 24, 26 penetrate the membrane that extends over the obturator foramen OF and the pubic arms 28, 30 are located anterior the pubic bone PB and are tunneled subcutaneously in an overlapping manner. In general, the support is implanted and aligned with the pubic symphysis PS. The strands 44 of the plication mechanism 32 are accessible through the perineal incision to allow the surgeon to take up slack or excess material that might exist in the support zone 42. Particularly as regards the male anatomy, the urethra/the penis describe a complex curvature and it is possible that an implanted support will present some level of undesirable bagginess as it is at times difficult to fit the two-dimensional structure of the support to the complex curvature of the anatomy.

FIG. 6 illustrates the plication mechanism 32 activated such that the support body 22 is in intimate contact with and fully supports the complex curvature of the anatomy. For example, strand 44 is tied into a knot to gather together the excess material of the support zone 42 tightly and in a supportive manner around the tissue surrounding urethra.

When the device 20 is implanted, the first and second pubic arms 28, 30 are tensioned, and pulling on the end portions 46, 48 of the plication mechanism 32 plicates the support zone 42 independent of the tensioned first and second pubic arms 28, 30 (that is, without changing the tension in the arms 28, 30).

When the device 20 is implanted, the first and second transobturator arms 24, 26 are tensioned, and pulling on the end portions 46, 48 of the plication mechanism 32 plicates the support zone 42 independent of the tensioned first and second transobturator arms 24, 26 (that is, without changing the tension in the arms 24, 26).

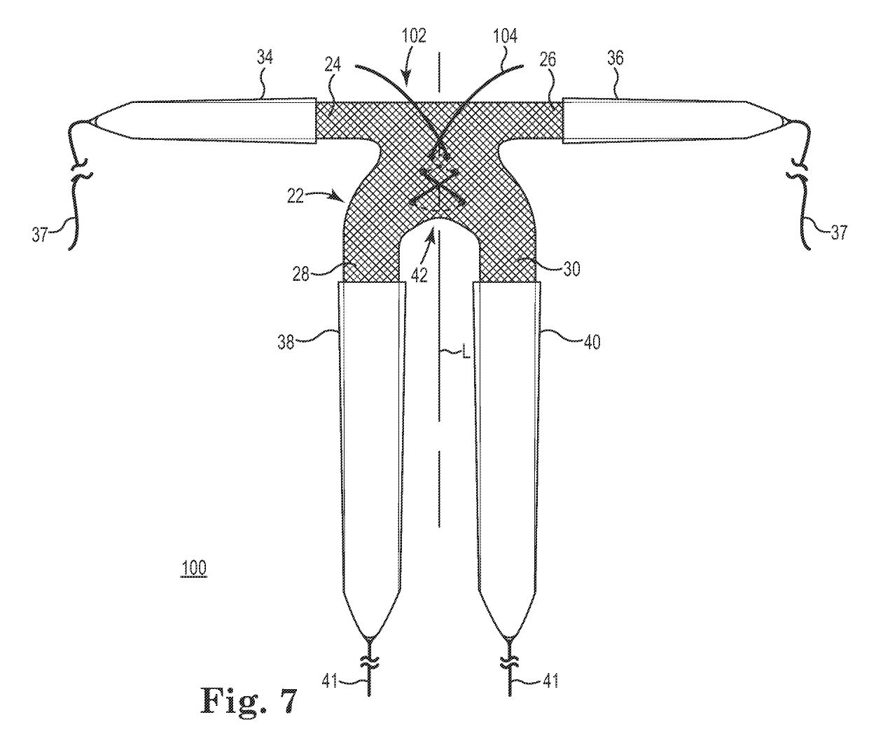

FIG. 7 is a top view of one embodiment of an incontinence treatment device 100 (the device 100). The device 100 includes the arms 24, 26 and 28, 30 described above that include their respective insertion sheaths 34, 36 and 38, 40. In one embodiment, a plication mechanism 102 is attached to the sport zone 42 and includes a strand 104 that is inserted through multiple pores of the porous material of the support body 22. In one embodiment, the strand 104 is laced through the support zone 42 and segments of the strand 104 cross over each other as illustrated. The lacing of the strand 104 in the support body 22 allows the plication mechanism 102 to apply more and more even tension to the support body 22 without changing the tension in the implanted arms 24, 26 and 28, 30.

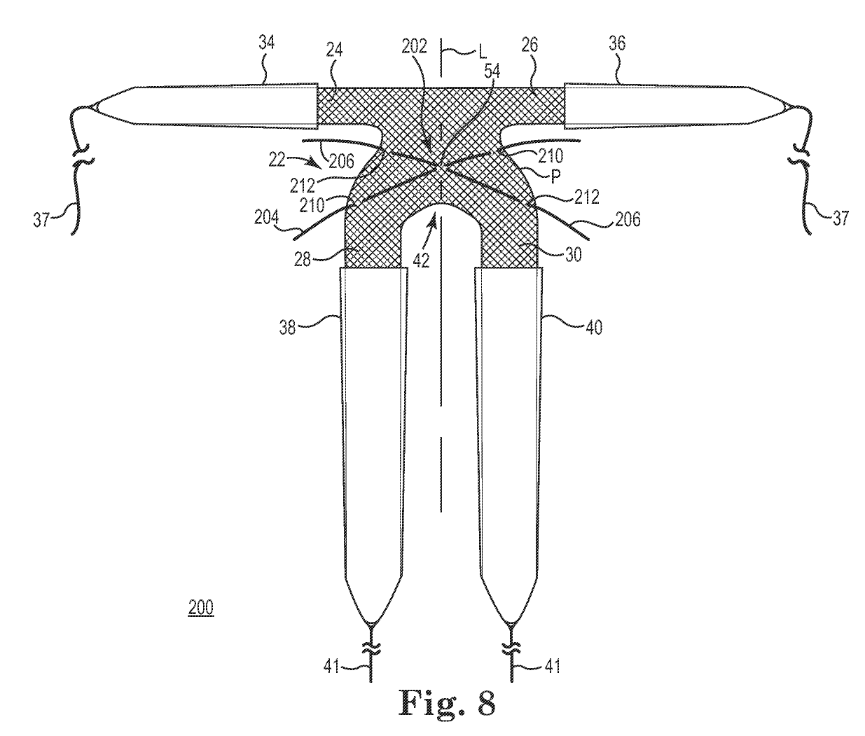

FIG. 8 is a top view of one embodiment of an incontinence treatment device 200 (device 200) including plication mechanism 202 attached to the support body 22. The device 200 includes the arms 24, 26 and 28, 30 described above in FIG. 1 and their respective insertion sheaths 34, 36 and 38, 40. In one embodiment, the plication mechanism 202 includes a first suture 204 and a second suture 206. Each of the sutures 204, 206 are attached to a perimeter P of the support zone 42 and at a location in the central region 54 of the support zone 42. For example, the suture 204 is attached or stitched through pores of the support body 22 at two perimeter P locations 210 and through the pores in the central region 54 of the support zone 42, and the suture 206 is attached or stitched through pores of the support body 22 at two perimeter P locations 212 and through the pores in the central region 54 of the support zone 42. After implantation, tension applied to the sutures 204, 206 applies tension to the central region of the support body 22 to remove wrinkles or material that has undesirably gathered in the support body 22.

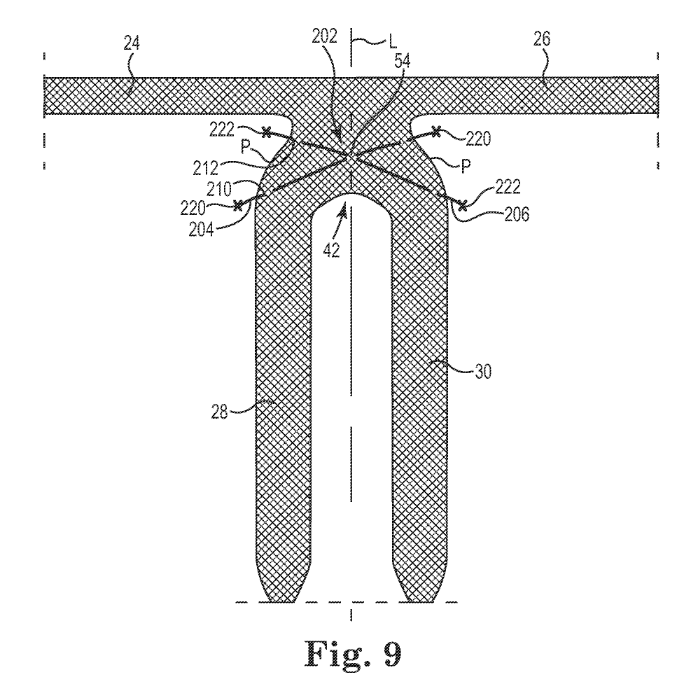

FIG. 9 is a schematic representation of one embodiment of the incontinence treatment device 200 illustrated in FIG. 8 with the arms 24, 26 and 28, 30 in an implanted configuration with the insertion sheaths removed.

During implantation of the incontinence treatment device, the surgeon attaches the suture 204 at the two perimeter P locations 210 to tissue of the periosteum (for example, or other suitable tissue) at locations 220 and the suture 206 at the two perimeter P locations 212 to tissue of the periosteum at locations 222. Each of the sutures 204, 206 is terminated at the respective locations 220, 222 or pulled to tighten against an anchoring suture placed at the periosteum locations 220, 222 to allow the plication mechanism 202 to remove slack in the support body 22 that can potentially gather in the support zone 42 inside the perimeter P. The sutures 204, 206 are suitably terminated at the periosteum locations 220, 222 after the surgeon acceptably removes the slack/wrinkles from the support body 22.

The support body is thus placed in contact with tissue around the urethra and tension applied to both of the first and second end portions of the suture 204 at locations 220 moves the first location of the support zone and the second location of the support zone to remove slack in the support body relative to the tissue around the urethra. In particular, the plication mechanism 202 is attached to the perimeter P of the support zone 42 and tension applied to both of the first and second end portions at 220 applies tension to the central region 54 of the support zone 42 of the support body 22.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of medical devices as discussed herein. Therefore, it is intended that this invention be limited only by the claims and the equivalents thereof.

What is claimed is:

1. A method of treating urinary incontinence, the method comprising:
forming an incision and accessing tissue around a urethra of a patient;
passing a first transobturator arm of an incontinence treatment device through the incision and through a first obturator foramen of the patient and a second transobturator arm of the incontinence treatment device through the incision and through a second obturator foramen of the patient;
passing a first pubic arm of the incontinence treatment device through the incision to a location anterior a pubic bone of the patient and a second pubic arm of the incontinence treatment device through the incision to a location anterior a pubic bone of the patient;
applying tension to the first and second transobturator arms and the first and second pubic arms of the incontinence treatment device and tensioning a urethral support body of the incontinence treatment device against the tissue around the urethra of the patient; and
pulling strands of a plication mechanism that is attached to the urethral support body and taking up slack in the urethral support body relative to the tissue around the urethra of the patient.

2. The method of claim 1, further comprising:
removing an insertion sheath from each of the first and second transobturator arms.

3. The method of claim 1, comprising utilizing a device to separately engage a suture attached to each of the first and second transobturator arms and passing the first and second transobturator arms through a respective one of the first and second obturator foramen.

4. The method of claim 1, wherein passing a first pubic arm of the incontinence treatment device through the incision to a location anterior a pubic one of the patient comprises passing a first pubic arm of the incontinence treatment device through the incision and subcutaneously to a location anterior a pubic bone of the patient.

5. The method of claim 1, wherein pulling strands of a plication mechanism comprises tying the strands of the plication mechanism together.

6. The method of claim 1, wherein pulling strands of a plication mechanism comprises placing tension on strands that are laced through multiple openings formed in the support body.

7. The method of claim 1, wherein a first strand of the plication mechanism is engaged with a first location of the support body and a second strand of the plication mechanism is engaged with a second location of the support body and pulling strands of the plication mechanism comprises moving the first and second locations in a medial direction.

8. The method of claim 1, wherein pulling strands of a plication mechanism comprises increasing tension of the urethral support body without changing tension in the first and second transobturator arms.

9. A method of treating urinary incontinence, the method comprising:
providing an incontinence treatment device having a support body with a transobturator arm and a pubic arm extending from the support body and a plication mechanism attached to the support body;
forming art incision and accessing tissue around a urethra of a patient;
passing a first transobturator arm of the incontinence treatment device through the incision and through a first obturator foramen of the patient and a second transobturator arm of the incontinence treatment device through the incision and through a second obturator foramen of the patient;
passing a first pubic arm of the incontinence treatment device through the incision to a location anterior a pubic bone of the patient and a second pubic arm of the incontinence treatment device through the incision to a location anterior a pubic bone of the patient;
applying tension to the first and second transobturator arms and the first and second pubic arms of the incontinence treatment device and tensioning the support body of the incontinence treatment device against the tissue around the urethra of the patient; and
activating the plication mechanism and removing slack between the support body and the tissue around the urethra of the patient.

10. The method of claim 9, further comprising:
removing an insertion sheath from each of the first and second transobturator arms.

11. The method of claim 9, comprising utilizing a device to separately engage a suture attached to each of the first and second transobturator arms and passing the first and second transobturator arms through a respective one of the first and second obturator foramen.

12. The method of claim 9, wherein passing a first pubic arm of the incontinence treatment device through the incision to a location anterior a pubic bone of the patient comprises passing a first pubic arm of the incontinence treatment device through the incision and subcutaneously to a location anterior a pubic bone of the patient.

13. The method of claim 9, wherein activating the plication mechanism comprises pulling strands of a plication mechanism.

14. The method of claim 13, wherein a first strand of the plication mechanism is engaged with a first location of the support body and a second strand of the plication mechanism is engaged with a second location of the support body and pulling strands of the plication mechanism comprises moving the first and second locations in a medial direction.

15. The method of claim 13, wherein pulling strands of a plication mechanism comprises placing tension on strands that are laced through multiple openings formed in the support body.

16. The method of claim 13, wherein pulling strands of a plication mechanism comprises increasing tension of the urethral support body without changing tension in the first and second transobturator arms.

17. The method of claim 9, wherein activating the plication mechanism comprises tying strands of the plication mechanism together.

18. The method of claim 9, further comprising:
centering the support body relative to the urethra of the patient by aligning a visual indicator provided by the plication mechanism with the urethra of the patient.
